(19) United States
(12) Patent Application Publication
(10) Pub. No.: US 2019/0282826 A1
(43) Pub. Date: Sep. 19, 2019

(54) DEVICES, SYSTEMS AND METHODS FOR PHOTOTHERAPY OF TREATMENT SITES

(71) Applicant: KLOX TECHNOLOGIES LIMITED, Dublin (IE)

(72) Inventor: Carlo BELLINI, Mont-Royal (CA)

(21) Appl. No.: 16/461,042

(22) PCT Filed: Nov. 9, 2017
(86) PCT No.: PCT/CA2017/051333

§ 371 Date: May 15, 2019

Related U.S. Application Data
(60) Provisional application No. 62/422,957, filed on Nov. 16, 2016.

(51) Int. Cl. A61N 5/06

(52) U.S. Cl. CPC A61N 5/062; A61N 5/0603; A61N 5/0614; A61N 5/0616; A61N 5/0624; A61N 2005/0606; A61N 2005/0662; A61N 2005/0634; A61N 2005/0637; A61N 2005/0642; A61N 2005/0659; A61N 2005/0661; A61N 2005/0626

(57) ABSTRACT

The present technology relates to a configurable phototherapeutic device or system. The phototherapeutic device or system comprises a plurality of light emitting devices adaptably connected to one another or to a support structure. The plurality of light emitting devices is configured for providing concurrent phototherapy to a plurality of anatomical features of a subject. The configurable phototherapeutic device or system also comprises a central controller which is operatively connected to the light emitting devices and each one of the light emitting devices being communicatively connected to the central controller. The central controller is configured for selectively causing at least one of the light emitting devices to emit light for phototherapy of at least one anatomical feature of the subject.

### CROSS-REFERENCE TO RELATED APPLICATION

The present application claims the benefit of and priority to U.S. provisional patent application No. 62/422,975, filed on Nov. 16, 2016; the content of this U.S. provisional patent application if herein incorporated in entirety by reference.

### TECHNICAL FIELD

The present technology relates generally to phototherapy and, more particularly, to devices, systems and methods for treatment of subjects (e.g., humans and animals) using controlled application of light.

### BACKGROUND INFORMATION

Phototherapy is used for a variety of healthcare (including medical and dental), cosmetic, and other purposes. Phototherapy can be used, for example, to promote wound healing, whiten teeth, kill bacteria, promote skin rejuvenation, treat skin conditions such as acne, even skin tone, and/or reduce or prevent scarring.

Various phototherapeutic devices have been developed, however some drawbacks have been observed with these devices. Some of these phototherapeutic devices may not be readily adaptable to various treatment settings, such as for providing different treatments to multiple anatomical features of a patient. Further, in some treatment settings where the phototherapeutic device is not also readily configurable or adaptable to external factors, such as the position or shape of the patient, the delivery of the phototherapy may not be constant and/or may not be optimal.

As such, there remains a need for improvements to address these as well as other drawbacks and to facilitate use and/or treatment of subjects with such phototherapeutic devices.

### SUMMARY OF DISCLOSURE

It is an object of the present technology to ameliorate at least some of the inconveniences present in the prior art.

According to an aspect of the present technology, there is provided a configurable phototherapeutic device comprising: a support structure; a plurality of light emitting devices adaptably connected to the support structure, the plurality of light emitting devices being configured for providing concurrent phototherapy to a plurality of anatomical features of a subject, each light emitting device of the plurality of light emitting devices comprising at least one light source configured to emit light adapted for phototherapy; and a central controller operatively connected to the plurality of light emitting devices, each one of the plurality of light emitting devices being communicatively connected to the central controller, the central controller being configured for selectively causing at least one of the plurality of light emitting devices to emit light for phototherapy of at least one anatomical feature of the plurality of anatomical features of the subject, the support structure being adapted for directing the light emitted by the at least one of the plurality of light emitting devices toward the at least one anatomical feature of the subject.

According to an aspect of the present technology, there is provided a configurable phototherapeutic device comprising: a plurality of light emitting devices configured for providing concurrent phototherapy to a plurality of anatomical features of a subject, each one of the plurality of light emitting devices comprising at least one light source configured to emit light adapted for phototherapy, each one of the plurality of light emitting devices being connected to at least one other of the plurality of light emitting devices; and a central controller operatively connected to the plurality of light emitting devices, each one of the plurality of light emitting devices being communicatively connected to the central controller, the central controller being configured for selectively causing at least one of the plurality of light emitting devices to emit light for phototherapy of at least one anatomical feature of the plurality of anatomical features of the subject.

According to an aspect of the present technology, there is provided the use of the configurable phototherapeutic device herein in phototherapy of a subject in need thereof.

According to an aspect of the present technology, there is provided the use of the configurable phototherapeutic device as defined herein, in phototherapy of one or more treatment sites of a subject in need thereof.

According to an aspect of the present technology, there is provided the use of the configurable phototherapeutic device as defined herein, in phototherapy of one or more anatomical features of a subject in need thereof.

According to an aspect of the present technology, there is provided the use of the configurable phototherapeutic device as defined herein, in a method of treatment of a subject, wherein the method of treatment comprises phototherapy.

According to an aspect of the present technology, there is provided a method for phototherapy of one or more treatment sites on a subject, said method comprising: applying one or more photoactivatable compositions to the one or more treatment sites; and exposing the applied one or more photoactivatable compositions to light emitted by the configurable phototherapeutic device as defined herein to activate the one or more photoactivatable compositions; wherein activation of the one or more photoactivatable compositions allow treatment of the one or more treatment sites.

According to an aspect of the present technology, there is provided a method for phototherapy of one or more treatment sites on a subject, said method comprising: applying one or more photoactivatable compositions to the one or more treatment sites; and exposing the applied one or more photoactivatable compositions to light emitted by the configurable phototherapeutic device as defined herein to activate the one or more photoactivatable compositions; wherein activation of the one or more photoactivatable compositions allow treatment of the one or more treatment sites.

According to an aspect of the present technology, there is provided a configurable phototherapeutic system comprising: a support structure; a plurality of light emitting devices adaptably connected to the support structure, the plurality of light emitting devices being configured for providing concurrent phototherapy to a plurality of anatomical features of a subject, each light emitting device of the plurality of light emitting devices comprising at least one light source configured to emit light adapted for phototherapy; and a central controller operatively connected to the plurality of light emitting devices, each one of the plurality of light emitting devices being communicatively connected to the central controller, the central controller being configured for selectively causing at least one of the plurality of light emitting devices to emit light for phototherapy of at least one anatomical feature of the plurality of anatomical features of the subject, the support structure being adapted for directing the light emitted by the at least one of the plurality of light emitting devices toward the at least one anatomical feature of the subject.

According to an aspect of the present technology, there is provided a configurable phototherapeutic system comprising: a plurality of light emitting devices configured for providing concurrent phototherapy to a plurality of anatomical features of a subject, each one of the plurality of light emitting devices comprising at least one light source configured to emit light adapted for phototherapy, each one of the plurality of light emitting devices being connected to at least one other of the plurality of light emitting devices; and a central controller operatively connected to the plurality of light emitting devices, each one of the plurality of light emitting devices being communicatively connected to the central controller, the central controller being configured for selectively causing at least one of the plurality of light emitting devices to emit light for phototherapy of at least one anatomical feature of the plurality of anatomical features of the subject.

According to an aspect of the present technology, there is provided the use of the configurable phototherapeutic system as defined herein in phototherapy of a subject in need thereof.

According to an aspect of the present technology, there is provided the use of the configurable phototherapeutic system as defined herein, in phototherapy of one or more treatment sites of a subject in need thereof.

According to an aspect of the present technology, there is provided a use of the configurable phototherapeutic system as defined herein, in phototherapy of one or more anatomical features of a subject in need thereof.

According to an aspect of the present technology, there is provided a use of the configurable phototherapeutic system as defined herein, in a method of treatment of a subject, wherein the method of treatment comprises phototherapy.

According to an aspect of the present technology, there is provided a method for phototherapy of one or more treatment sites on a subject, said method comprising: applying one or more photoactivatable compositions to the one or more treatment sites; and exposing the applied one or more photoactivatable compositions to light emitted by the configurable phototherapeutic system as defined herein to activate the one or more photoactivatable compositions; wherein activation of the one or more photoactivatable compositions allow treatment of the one or more treatment sites.

According to an aspect of the present technology, there is provided a method for phototherapy of one or more treatment sites on a subject, said method comprising: applying one or more photoactivatable compositions to the one or more treatment sites; and exposing the applied one or more photoactivatable compositions to light emitted by the configurable phototherapeutic system as defined herein to activate the one or more photoactivatable compositions; wherein activation of the one or more photoactivatable compositions allow treatment of the one or more treatment sites.

All documents referred to herein are incorporated by reference in their entirety. Should there be contradictions between the definitions of terms provided in documents incorporated herein by reference and definitions of such terms provided in the present application, the definitions in the present application prevail.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of embodiments of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 illustrates a configurable phototherapeutic device for performing phototherapy treatment on a subject, in accordance with an embodiment of the present technology;

FIG. 2 illustrates the subject being exposed to light emitted by the phototherapeutic device of FIG. 1, according to one possible arrangement of the configurable phototherapeutic device of FIG. 1;

FIG. 3 illustrates the subject being exposed to light emitted by the configurable phototherapeutic device of FIG. 1, according to another possible arrangement of the configurable phototherapeutic device of FIG. 1;

FIG. 4 is a schematic illustration of components of the configurable phototherapeutic device of FIG. 1;

FIG. 5 illustrates an area of the subject being exposed to light emitted by the configurable phototherapeutic device of FIG. 1;

FIG. 6 illustrates a photoactivatable composition for treating the subject that can be exposed to the light emitted by the configurable phototherapeutic device of FIG. 1;

FIG. 7 is a perspective view of a lamp of the configurable phototherapeutic device of FIG. 1, with a portion of a housing removed to show internal components;

FIG. 8A is a top plan view of the lamp of FIG. 7;

FIG. 8B is a bottom plan view of the lamp of FIG. 7;

FIG. 9 is a schematic illustration of components of the lamp of FIG. 7;

FIG. 10 is a schematic illustration of a light source of the lamp of FIG. 7;

FIG. 11 is a schematic illustration of a control system of the lamp of FIG. 7;

FIG. 12 is a schematic illustration of a controller of the control system of FIG. 11;

FIG. 13 is a schematic illustration of sensors of the control system of FIG. 11;

FIG. 14 is a schematic illustration of a power supply of the control system of FIG. 11;

FIG. 15 is a schematic illustration of an interface of the control system of FIG. 11;

FIG. 16 is a schematic illustration of a cooling system of the lamp of FIG. 7;

FIG. 17 is a configurable phototherapeutic device for performing phototherapy treatment on a subject, in accordance with another embodiment of the present technology;

FIG. 18 is a configurable phototherapeutic device for performing phototherapy treatment on a subject, in accordance with yet another embodiment of the present technology;

FIG. 19 is a configurable phototherapeutic device for performing phototherapy treatment on a subject, in accordance with yet another embodiment of the present technology; and

FIG. 20 is a flow chart illustrating a method of using the configurable phototherapeutic device of FIG. 1.

It is to be understood that the description and drawings are only for the purpose of illustrating certain embodiments of the present disclosure and are an aid for understanding. They are not intended to be a definition of the limits of the disclosure. Unless otherwise noted, the figures are not drawn to scale.

### DETAILED DESCRIPTION

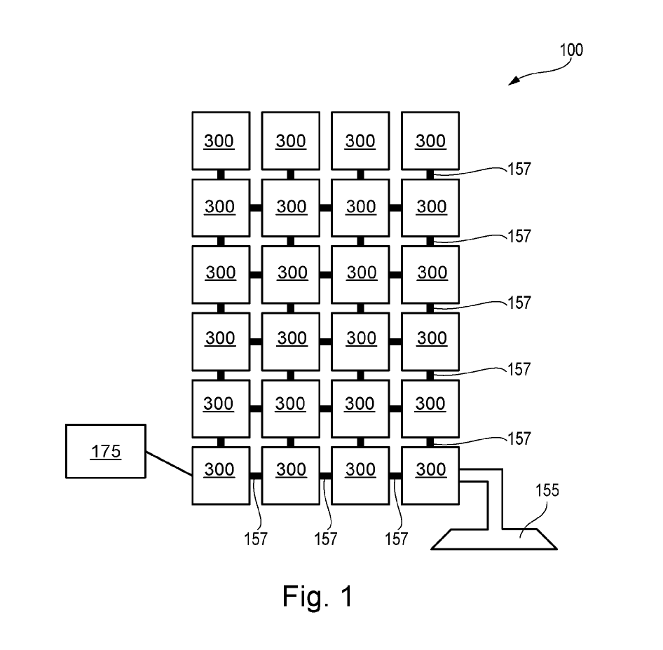

FIGS. 1 to 3 illustrate a non-limiting example of a configurable phototherapeutic device or system 100 for performing phototherapy treatment on a subject 200 in accordance with an embodiment of the present disclosure. In this example, the subject 200 is a human. It is contemplated that the subject 200 could be an animal. As will be described in detail herein, the configurable phototherapeutic device or system 100 can be configured to provide concurrent, different phototherapy treatments to one or more anatomical features of the subject 200. In some embodiments of the configurable phototherapeutic device or system 100, the device 100 can further be adaptable to the contour of the one or more anatomical features of the subject 200 receiving the phototherapy treatment.

Although the configurable phototherapeutic device or system 100 will be referred to herein generally as the device 100, it is contemplated that the configurable phototherapeutic system 100 includes the configurable phototherapeutic device 100.

In some embodiments, the configurable phototherapeutic system 100 could further include one or more photoactivatable compositions 600.

In some embodiments, the configurable phototherapeutic system 100 could further include one or more elements to aid in configurable phototherapy, including, but not limited to, a protective housing, a subject-receiving bed, and one or more ground-engaging elements for system mobility.

The configurable phototherapeutic device 100 comprises a plurality of light emitting devices 300, referred to herein as lamps 300, to emit light for concurrently treating one or more treatment sites of the subject 200, as will be described in more detail below. In order to implement the phototherapeutic treatment, the phototherapeutic device 100 is generally used in combination with a photoactivatable composition 600, of which at least a portion may be applied on the subject 200, also described in more detail below. It is contemplated that in some embodiments of the configurable phototherapeutic device 100, the phototherapeutic treatment could be differently implemented.

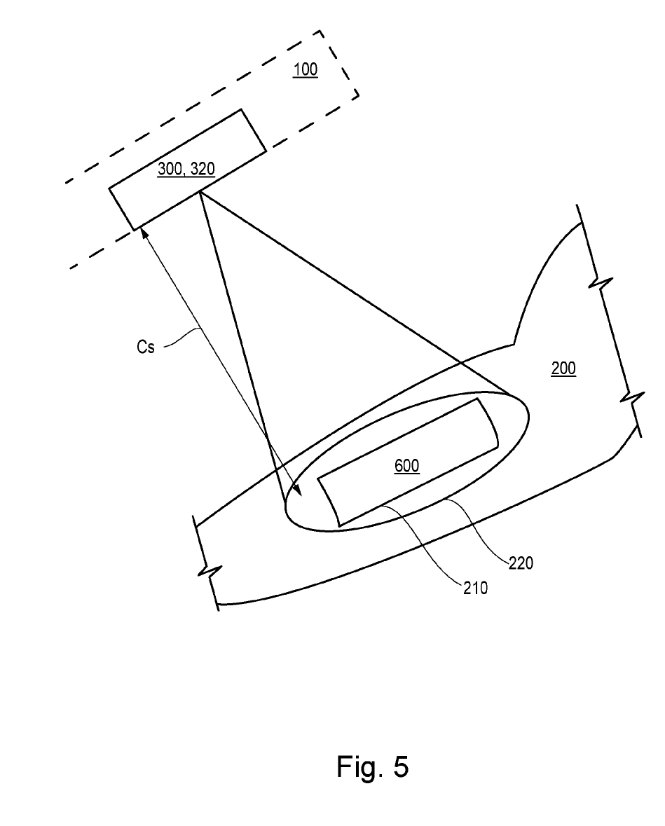

Each lamp 300 comprises a light source 320 to emit light for the phototherapy treatment on the subject 200. Specifics of the light sources 320 will be discussed below. As illustrated in FIG. 5, the light emitted by the light source 320 of one lamp 300 irradiates an area 220 which intersects a treatment site 210 of the subject 200 that is covered by the photoactivatable composition 600. The configurable phototherapeutic device 100 can be used for healthcare (including medical and/or dental), personal care, cosmetic, animal care and/or other purposes. For instance, in various examples of implementation, the configurable phototherapeutic device 100 can be used, for example, to: treat a wound (e.g., to help healing); provide an antibacterial, antiviral, antifungal and/or anti-parasitic treatment; treat an inflammation; treat skin (e.g., rejuvenate the skin's appearance; treat a skin condition such as acne, psoriasis, rosacea, dermatitis, eczema, or the like) and soft tissues; prevent the appearance of a skin condition such as acne, psoriasis, rosacea, dermatitis, eczema, or the like; even the skin's tone; reduce, eliminate or prevent scarring; and prevent and/or treat oral and/or dental conditions.

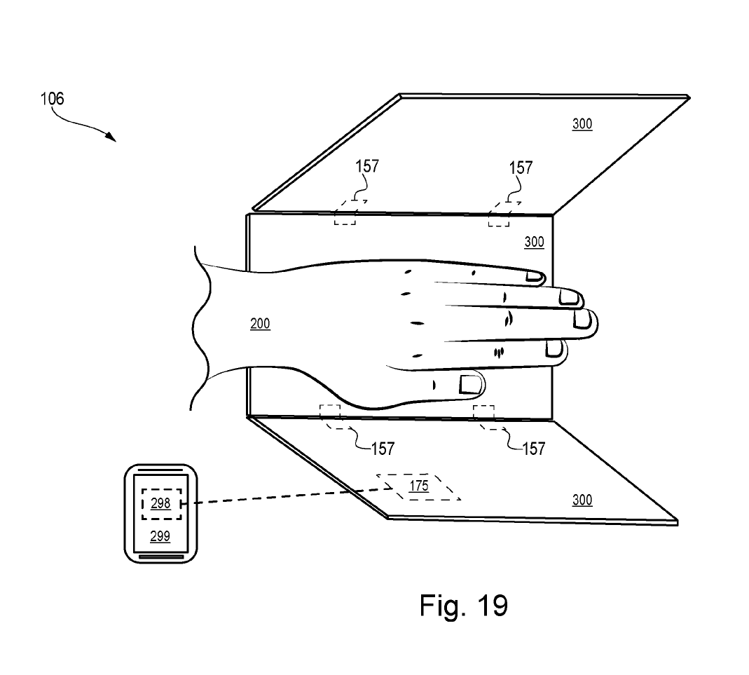

The configurable phototherapeutic device 100 can be used by a user, who may be, for example, a healthcare practitioner such as for example, a clinician (e.g., a physician, a nurse, a medical technician, a dentist, a dental assistant, or any other healthcare practitioner), a cosmetic practitioner, a veterinary, the subject 200 himself/herself, or any other suitable individual. The configurable phototherapeutic device 100 may be used in a residence, a healthcare facility (e.g., a hospital, a medical or dental clinic, or the like), a cosmetic facility, a veterinary clinic and/or any other suitable setting. It is also contemplated that the configurable phototherapeutic device 100 could be implemented as a mobile, over-the-counter device, such that the device could be operated and used by the subject 200 himself/herself at their own residence, as will be described below with respect to FIG. 19.

The non-limiting example of the configurable phototherapeutic device 100 comprises twenty-four lamps 300. Different embodiments of configurable phototherapeutic devices may have more or less lamps 300, depending on the specific embodiment. Some embodiments of the configurable phototherapeutic device 100 may have as few as two or three lamps (see for example FIG. 19), while it is also contemplated that some embodiments may have a large number of lamps 300 (as is illustrated in FIG. 1).

In some embodiments, one or more of the lamps 300 could be detachably connected to each other such that the lamps 300 are selectively disconnectable. In this embodiment, each lamp 300 generally contains within itself the components necessary for its operation, as will be discussed in greater detail below. It is also contemplated that none or only a portion of the lamps 300 could be selectively disconnectable.

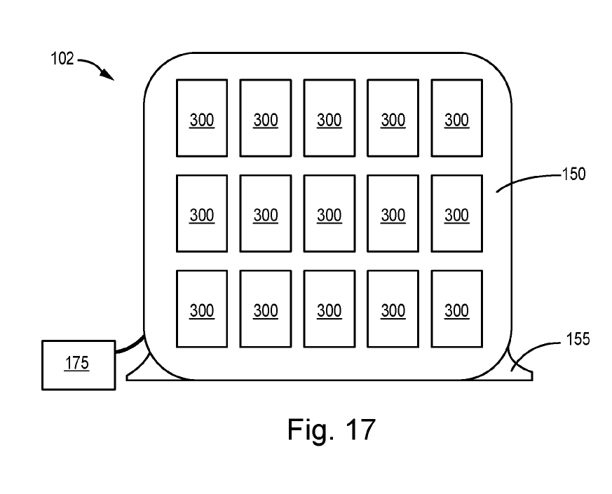

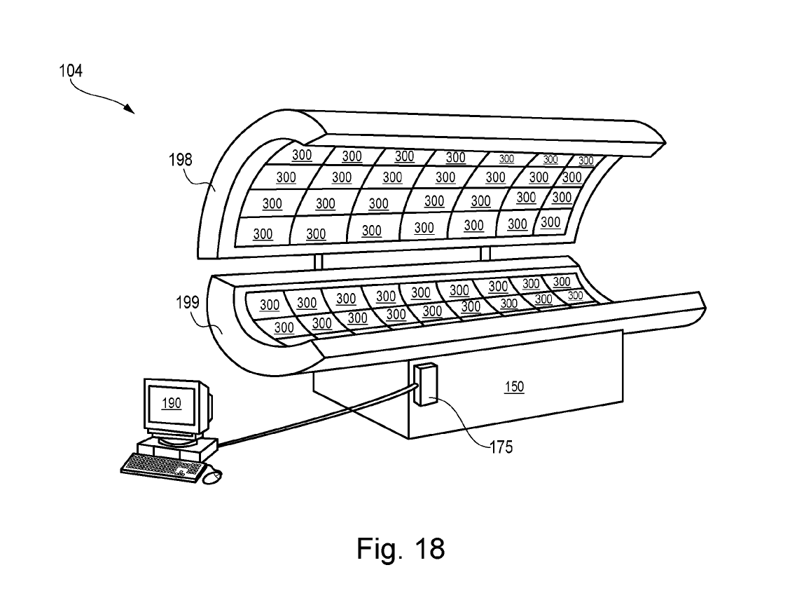

Each lamp 300 of the configurable phototherapeutic device 100 is adjustably connected to at least one other lamp 300 by a connecting member 157. Some of the lamps 300 are connected to only one other lamp 300 via one connecting member 157, some lamps 300 to two other lamps 300 via two connecting members 157, some lamps 300 to three other lamps 300, while other lamps 300 are connected to four other lamps 300. The number of connecting members 157 present in any given embodiment of the configurable phototherapeutic device 100 will vary depending on the number and arrangement of the lamps 300 and could vary depending on other factors (a specific application for which an embodiment of the configurable phototherapeutic device 100 could be designed, for example). It is also contemplated that the lamps 300 could be adjustably connected directly to one another without aid of the connecting members 157. It is further contemplated that in some embodiments, the lamps 300 could be adjustably connected to a support structure, rather than being connected to one another; such examples will be described below with respect to FIGS. 17 and 18.

The connecting members 157 are rigid members pivotably and rotationally connected at each end to one of the lamps 300. The lamps 300 thus may be rotated, pivoted, tilted, laterally shifted or displaced with respect to one another in order to adjust the orientation of the lamps 300 to direct light emitted by the lamps 300 toward the one or more anatomical features of the subject 200. While in the embodiment of FIGS. 1 to 3 all of the lamps 300 have adjustable orientations, it is contemplated that in some embodiments, only a portion of the lamps 300 may have an adjustable orientation. One or more of the connecting members 157 are connected to a base 155 for supporting the configurable phototherapeutic device 100, shown schematically in FIG. 1.

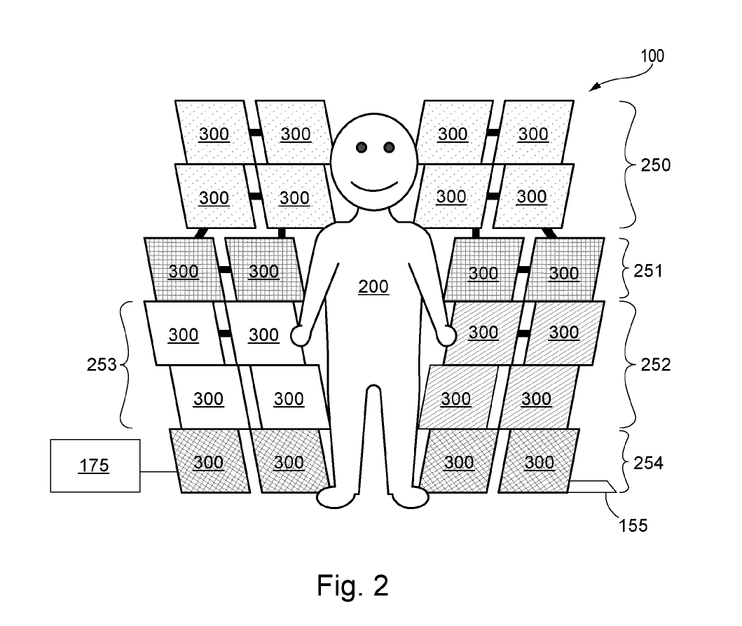

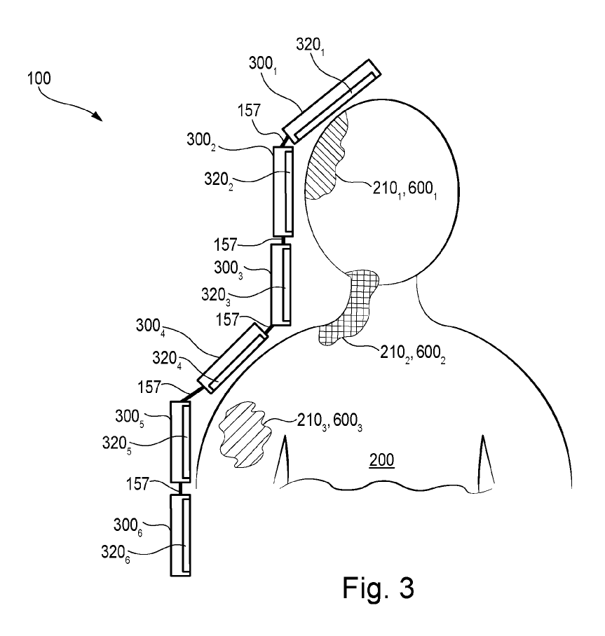

FIGS. 2 and 3 illustrate two examples of arrangements of the configurable phototherapeutic device 100 of FIG. 1, where the lamps 300 of the configurable phototherapeutic device 100 have been spatially adjusted in order to direct light emitted from the lamps 300 toward different anatomical features of the subject 200 for phototherapy. In FIG. 2, the lamps 300 have been rotated, angled, and displaced with respect to one another in order to properly illuminate different anatomical features of the whole body of the subject 200, and thus to aim light emitted from the lamps 300 toward different treatment areas. In FIG. 3, the lamps 300 have been rotated, angled, and displaced with respect to one another in order to properly illuminate the head, neck, and torso regions of the subject 200 for phototherapy of those regions.

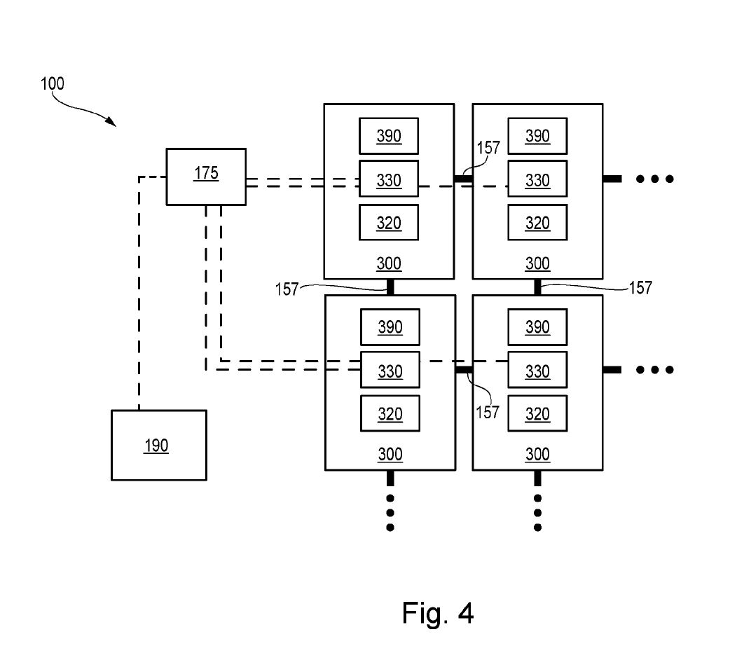

The configurable phototherapeutic device 100 further comprises a central controller 175, operatively and communicatively connected to each of the lamps 300, as is shown schematically in FIG. 4. The operative and communicative connection between the central controller 175 and the lamps 300 and/or other components of the device 100 could be implemented in several ways, including, but not limited, a direct electrical, cable connection and a wireless connection. The central controller 175 is configured to selectively cause one or more of the lamps 300 to emit light for phototherapy, and to control operation of the lamps 300, as will be described below. The central controller 175 manages operation of the configurable phototherapeutic device 100, based on instructions stored to a computer based memory and/or instructions received from the operator and can be implemented using any suitable hardware or software.

The central controller 175 could be implemented, for example, as a computer chip or a computer board or card internal to the configurable phototherapeutic device 100. In some embodiments, the central controller 175 could be implemented as an integrated or stand-alone computational device, such as a personal computer, configured to control the lamps 300 based on instructions in memory of that computational device and/or received through an input device from the operator. In some embodiments, the central controller 175 could include various connectivity means or capabilities, including but not limited to: Wi-Fi™ (wireless local area network) connectivity and blue-tooth connectivity. In some embodiments, the central controller 175 could include hardware, such as programmable chips and memory systems, necessary to render the central controller 175 fully programmable. The central controller 175 could also be implemented as software on a device external and operatively connected to the configurable phototherapeutic device 100, configured to operate the configurable phototherapeutic device 100. It is further contemplated that the central controller 175 could be implemented, or controlled by, an application on an external electronic device, as will be described below with respect to FIG. 19.

In some embodiments, the central controller 175 could include, or be connected to, an external display 190 for displaying information to the user or operator of the configurable phototherapeutic device 100. It is further contemplated that the central controller 175 could provide an interface for receiving instructions from the user or operator, such as a graphical user interface (GUI). In some embodiments, the central controller 175 and/or the external display 190 could include touch-screen control for operating the configurable phototherapeutic device 100. In some further embodiments, the user or operator could use the central controller 175 and/or the external display 190 and/or the touch-screen control to implement updates to software for operating the configurable phototherapeutic device 100. In some further embodiments, the updates could be acquired wirelessly.

The central controller 175 selectively controls the lamps 300, such that one or more of the lamps 300 can be activated and caused to emit light for phototherapy. Further, the central controller 175 is configured to control different properties of light emitted from the lamps 300, including, but not limited to, the peak wavelength of emitted light, average power density, spectral width, pulse width and/or duration, and fluence. It is also contemplated that the central controller 175 could provide simplified controls, such that the user would control only a subset of the possible control variables of the lamps 300. For one non-limiting example, the central controller 175 may provide the user the ability to control only power output, timing of the power, and/or power modulation. In some embodiments, controls provided by the central controller and/or an application to operate the configurable phototherapeutic device 100 may be highly simplified and designed such that a child could operate the configurable phototherapeutic device 100.

The central controller 175 is configured to provide one or more phototherapeutic patterns of emitted light for providing concurrent phototherapy to the one or more anatomical features of the subject 200. The central controller 175 can do this by selectively activating one or more subsets of the lamps 300 according to which anatomical features of the subject 200 are to undergo treatment. The phototherapeutic patterns will be discussed in more detail below.

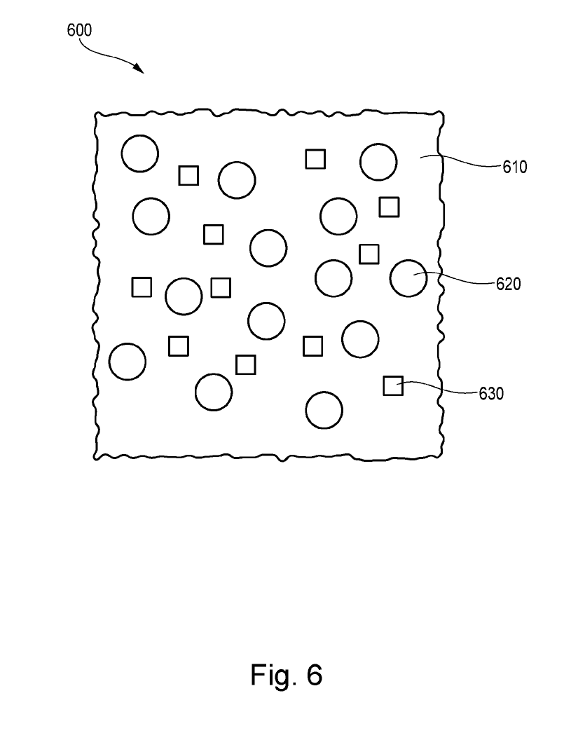

With reference to FIGS. 3, 5 and 6 and as mentioned above, in order to implement the phototherapeutic treatment, the configurable phototherapeutic device 100 may be used in combination with the photoactivatable composition 600 whereby the photoactivatable composition 600 is illuminated by the light emitted by the lamps 300 to treat the subject 200. One lamp 300 is shown for simplicity of explanation in FIG. 5, where the light emitted by one lamp 300 irradiates the area 220 which intersects the treatment site 210 of the subject 200, covered by the photoactivatable composition 600.

As is illustrated in FIG. 3, multiple lamps 300 of the configurable phototherapeutic device 100 can be used to illuminate one or more photoactivatable compositions 600 to concurrently treat the subject 200, according to the present technology. The subject 200 in this non-limiting example has three treatment sites 210_{X} on three different anatomical features needing phototherapy treatment: treatment site 210₁ on the head, treatment site 210₂ on the neck, and treatment site 210₃ on the right arm. If, for instance, the treatment site 210₁ on the head is acne to be treated, the treatment site 210₂ on the neck is a burn, and the treatment site 210₃ on the right arm is a chronic wound, the subject 200 may require three (or less) different treatments. In the present technology, rather than providing the different treatments successively (if possible), embodiments of the configurable phototherapeutic device 100 can enable the user or operator to provide these three distinct treatments concurrently. In addition, the configurable phototherapeutic device 100 is adaptable to the shape of each one of the three different treatment sites 210_{X} to be treated.

For the treatment site 210₁ on the head, the appropriate photoactivatable composition 600₁ for acne is applied to the treatment site 210₁. Similarly, for the treatment site 210₂ on the neck, the appropriate photoactivatable composition 600₂ for a burn is applied thereto. Finally, for the treatment site 210₃ on the arm, the appropriate photoactivatable composition 600₃ for a chronic wound is applied thereto. In some implementations of this embodiment, a single photoactivatable composition 600 may be used for treatment of more than one (or all) of the treatment sites 210_{X} to be treated. In such implementations, the photoactivatable composition 600 may comprise the necessary components for treatment of treatment sites 210_{X} to be treated.

The lamps 300 of the configurable phototherapeutic device 100 have been rotated, tilted, and shifted to properly align with the head, neck and arm of the subject 200, as described above. Only six of the lamps 300 are illustrated for simplicity. The lamps 300 can then be selectively controlled by the central controller 175 to emit the light appropriate for each of the three treatment sites 210_{X} and their corresponding photoactivatable compositions 600_{X}. In this example, the light sources 320₁ and 320₂ of the lamps 300₁ and 300₂, aimed at the treatment site 210₁, produce light suitable for photoactivation of the photoactivatable composition 600₁. The light source 3203 of the lamp 300₃, aimed at the treatment site 210₂, produces light suitable for photoactivation of the photoactivatable composition 600₂. The light source 320₅ of the lamp 300₅, aimed at the treatment site 210₃, produces light suitable for photoactivation of the photoactivatable composition 600₃. In some implementations of this embodiment, the light sources 320₄ and 320₆ of the lamps 300₄ and 300₆, are not activated by the central controller 175 however, because they are not aligned with any treatment sites requiring phototherapy. In some other implementations of this embodiment, the light sources 320₄ and 320₆ of the lamps 300₄ and 300₆, are activated by the central controller 175 to provide a light that is not therapeutical to the anatomical features of the subject that do not require treatment.

Details of the photoactivatable compositions 600_{X} will now be discussed, with reference to photoactivatable composition 600 only, but it is contemplated that the details apply equally to any photoactivatable composition 600_{X} suitable for the present technology. The photoactivatable composition 600 may comprise a carrier 610 and a photoactivatable agent 620 which may be dispersed in the carrier 610. In this some embodiments, the photoactivatable composition 600 may also comprise an oxygen-providing agent 630.

The carrier 610 is a medium carrying the photoactivable agent 620. In this embodiment, the carrier 610 is a fluid, a liquid or a semi-liquid (e.g., a gel). More particularly, in this embodiment, the carrier 610 may comprise a diluent, a solvent, a thickening agent, a cross-linker, a stabilizer, a surfactant, an initiator, an antimicrobial, or the like.

The photoactivatable agent 620 may be implemented in any suitable way. Examples of the photoactivable agent 620 in some embodiments include xanthenes derivatives, azo dyes, biological stains, carotenoids, fluorescent dyes or stains, biological dyes, histological dyes, chlorophyll dyes, methylene blue dyes, food colorings, naturally occurring photoactive agents, or the like.

In some implementations, the photoactivatable agent 620 is a xanthene dye. The xanthene dye may be fluorescein or eosin, or any other xanthene dye. In some implementations, the photoactivatable agent 620 is Eosin Y. In some other implementations, the photoactivatable agent 620 is Fluorescein. In some other implementations, the photoactivatable agent 620 is Eosin Y and Fluorescein. In some implementations, the photoactivatable agent 620 is Rose Bengal. In some other implementations, the photoactivatable agent 620 is Eosin Y and Rose Bengal. In some other implementations, the chromophore is Eosin Y, Rose Bengal and Fluorescein.

The oxygen-providing agent 630 may be implemented in any suitable way. Examples of the oxygen-providing agents 630 in some embodiments may include various peroxides, such as hydrogen peroxide, carbamide peroxide, urea peroxide, benzoyl peroxide, peroxy acids, and the like.

As an alternative, in some embodiments, the photoactivatable agent 620 and the oxygen-providing agent 630 may be separated from each other, such that the photoactivatable agent 620 is included in the photoactivatable composition 600 while the oxygen-providing agent 630 is included in another photoactivatable composition (not illustrated) including a carrier in which is dispersed the oxygen-providing agent. In various examples of implementation, the carrier may be implemented as discussed above in respect of the carrier 610 of the photoactivatable composition 600. The configurable phototherapeutic device 100 is thus used in combination with a photoactivatable composition system that comprises the photoactivatable composition 600 and the other photoactivatable composition which cooperate by being intersected by a common light transmission path.

Examples of the photoactivatable composition 600 that may be used in various embodiments can be found, for example, in PCT/CA2009/001608; PCT/CA2009/001615; PCT/CA2010/001134: PCT/CA2013/000532; PCT/CA2013/000395; and PCT/US2013/058102, the entirety of each of which is incorporated herein by reference.

As mentioned above, the central controller 175 is configured to provide one or more phototherapeutic patterns of emitted light for providing concurrent phototherapy to multiple anatomical features of the subject 200 by selectively activating one or more subsets of the lamps 300, according to which anatomical features of the subject 200 are to undergo treatment. In the example of FIG. 3 described above, the configurable phototherapeutic device 100 was configured to be spatially adapted to the subject 200, as well as to produce light (or not produce light) in patterns appropriate to several concurrent phototherapy treatments.

As used herein, the phototherapeutic patterns simply represent the lamps 300 selected to emit light by the central controller 175 and properties of that emitted light. For one non-limiting example, the central controller 175 may activate all of the lamps 300 in the configurable phototherapeutic device 100, but with half of the lamps 300 emitting blue light, and the other half emitting green light. In such a case, the central controller 175 would be said to be providing two phototherapeutic patterns. As another non-limiting example, the central controller 175 may control all of the lamps 300 in the configurable phototherapeutic device 100 to emit blue light, but with half of the lamps 300 emitting the blue light at half the power density of the other half of the lamps 300. These are simply some non-limiting examples of a phototherapeutic pattern to be provided by the central controller 175. It is contemplated that other phototherapeutic patterns could include more or less than two colors of light. It is also contemplated that different light provided by phototherapeutic patterns could be split equally between different colors (e.g. half blue/half green as in the example above), or split unequally between different types of light (e.g. almost all blue, with a small amount of green light). It is further contemplated that the phototherapeutic patterns could provide light that is not in the visible spectrum, such as ultraviolet light or infrared light.

Depending on the specific embodiment and any given use of the configurable phototherapeutic device 100, the central controller 175 can be configured to produce one or more phototherapeutic patterns to provide concurrent phototherapy for one or more conditions of the subject 200. The central controller 175 could be configured to activate a first subset of the plurality of lamps 300 to provide a first phototherapeutic pattern and activate a second subset of the plurality of lamps 300 to provide a second phototherapeutic pattern. In some cases, the central controller 175 could further be configured to activate a third subset of the plurality of lamps 300 to provide a third phototherapeutic pattern, a fourth subset of lamps 300 to provide a fourth phototherapeutic pattern, and/or a fifth subset of the plurality of lamps 300 to provide a fifth phototherapeutic pattern. It is contemplated that the central controller 175 could direct the configurable phototherapeutic device 100 to produce more or less than five phototherapeutic patterns, depending on the specific subject 200, use, and/or embodiment.

In some cases, the different phototherapeutic patterns would be distinguished such that the lamps 300 grouped into each different pattern emit light of different wavelengths. Additionally or alternatively, the different phototherapeutic patterns would be distinguished such that the lamps 300 grouped into each different pattern emit light with different power densities of the light emitted. For example, a first subset of the lamps 300 could emit light with a first peak wavelength, a second subset of the lamps 300 could emit light with a second peak wavelength, and a third subset of the lamps 300 could emit light with a third peak wavelength. In some uses of any given configurable phototherapeutic device 100, the central controller 175 could further create patterns including a fourth subset of the lamps 300 emitting light with a fourth peak wavelength and a fifth subset of the lamps 300 emitting light with a fifth peak wavelength.

Generally, the configurable phototherapeutic device 100 will be controlled to produce different patterns depending on different treatment procedures and on which anatomical features are to undergo treatment. For example, in the case where the central controller 175 has activated two phototherapeutic patterns to treat the subject 200, the first phototherapeutic pattern will be in phototherapeutical alignment with a first anatomical feature of the subject and the second phototherapeutic pattern will in phototherapeutical alignment with a second anatomical feature of the subject. Such is the case in the example illustrated in FIG. 3, described above.

In the example illustrated in FIG. 2, the configurable phototherapeutic device 100 has been configured to adapt to the specific phototherapy treatment to be undergone by the subject 200. In this example, the subject 200 is undergoing phototherapy at different points over the whole body of the subject 200 (specific treatment regions not being depicted). In this example, the subject 200 is being exposed to five different phototherapeutic patterns, as defined by the subgroups 250-254 of the lamps 300. Each different group of lamps 300 could emit different “types” of light, where as before, the “types” of light may be differentiated based on a number of factors, including peak wavelength, average wavelength, bandwidth, intensity, or the like.

In the example illustrated in FIG. 3 and as generally discussed above, the configurable phototherapeutic device 100 produces three patterns for treating the three treatment sites 210₁, 210₂, and 210₃. It should be noted that the phototherapeutic patterns of the above examples are simply patterns activated by the central controller 175 in one example use of the configurable phototherapeutic device 100. Different patterns may be activated by the central controller 175 for any given use of the configurable phototherapeutic device 100, such that the light emitted can be configured to convey the appropriate one or more phototherapy treatments needed for each subject 200.

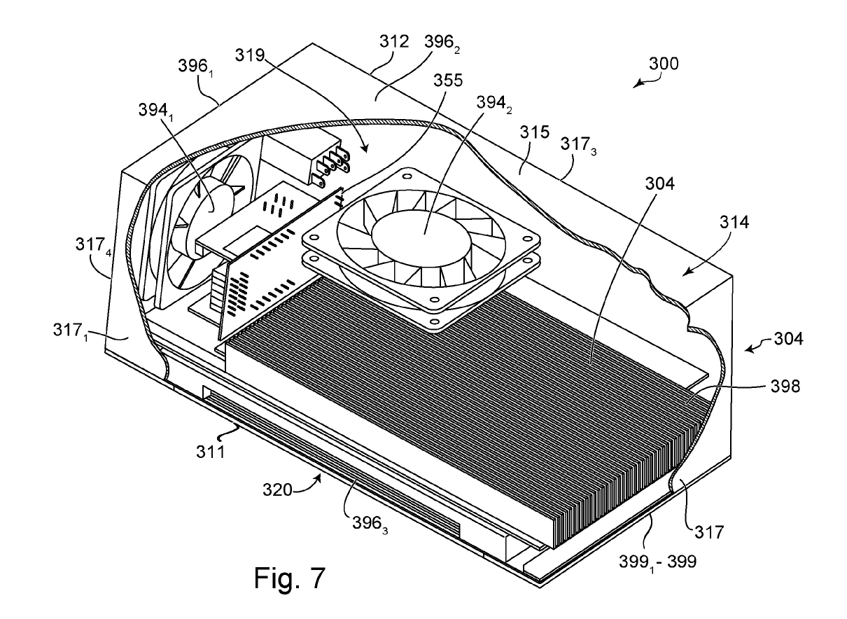

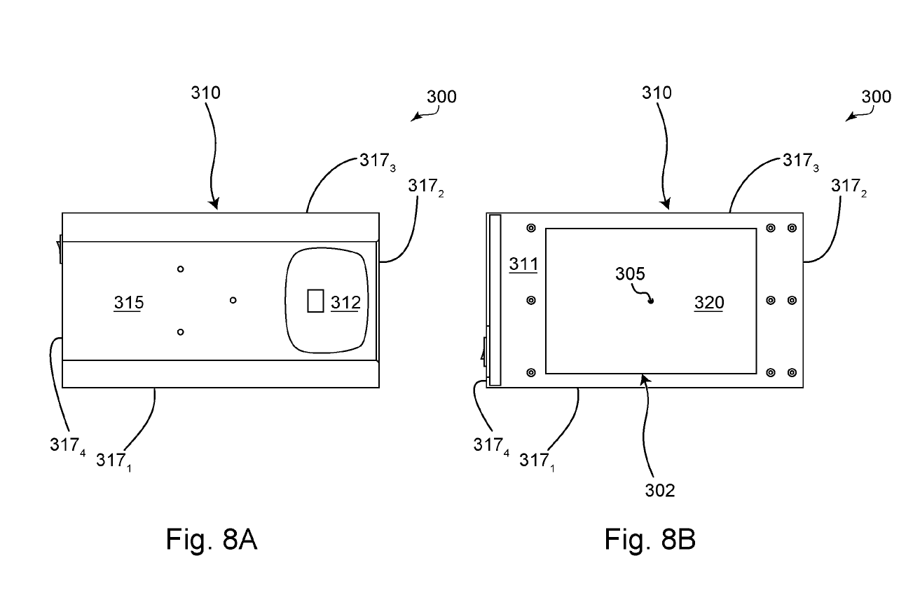

With reference to FIGS. 7 to 16, specific operational details of the lamps 300 according to one embodiment of the present technology will be discussed. It should be noted that orientations assigned to the lamps 300 herein (e.g. “top” and “bottom”) are simply chosen for simplicity of explanation and are inconsequential to operation of the lamps 300 or the phototherapeutic device 100. One embodiment of the lamps 300 is described in further detail in PCT Application PCT/CA2016/050544, filed 12 May 2015, the entirety of which is incorporated herein by reference. It should further be noted that the lamps 300 of the configurable phototherapeutic device 100 will be described below as all being the same in a given embodiment of the configurable phototherapeutic device 100, but it is contemplated that various embodiments of the lamps 300 could be included in the given embodiment of the configurable phototherapeutic device 100.

Each lamp 300 comprises a control system 330 for controlling operation of the light source 320 and a housing 312 supporting various components of the lamp 300, including the light source 320 and the control system 330. Also, in this embodiment, the lamp 300 comprises a cooling system 390 to manage heat generated by the light source 320 and the control system 330.

In this embodiment, the housing 312 defining a cavity 319 that houses various components of the lamp 300, including at least part of the light source 320, the control system 330, and the cooling system 390. It is contemplated that the housing 312 of all or a portion of the lamps 300 could also comprise handles for allowing the user to handle (e.g., move and/or hold) the lamp 300. The housing 312 may be composed of one or more of a variety of materials, including different varieties of plastics and metals, or any combination thereof.

The housing 312 may be implemented in any suitable way, taking on any suitable shape. The housing 312 comprises a periphery 314 which includes a top surface 315, a bottom surface 311, and lateral surfaces 317₁-317₄. For example, in some embodiments, the housing 312 may take on the shape of a rectangular box with a length, a width, and a height, where the magnitude of the length is larger than the magnitude of the width. In other embodiments, the housing 312 may be a cuneiform rectangular box, or may define a generally semi-spherical or semi-ovoidal shape. In some embodiments, the housing 312 may taper in a lengthwise direction or in a widthwise direction of the lamp 300. It is also contemplated that the housings 312 of different lamps 300 of the one embodiment of the phototherapeutic device 100 may have various different forms. Many other possible shapes of the housing 312 are also possible. The housing 312 may be composed of one or more of a variety of materials, including different varieties of plastics and metals, or any combination thereof. In some embodiments, the housing 312 may be wholly formed as a one-piece component, whereas in some other embodiments, it may be composed of a plurality of pieces fastened together (e.g., using an adhesive and/or one or more mechanical fasteners such as screws, bolts, rivet, or the like; by welding, or the like).

The light generated by the light source 320 is emitted at a light-emitting side 302 of the lamp 300 (the light source 320 will be described in more detail below). In some embodiments, the light-emitting side 302 of the lamp 300 comprises a protection layer (not shown) for safety of the subject to the treated and for protecting the light source 320 from any surrounding elements (e.g., microorganisms, particles, gases, liquids, solids, of the like) that may otherwise enter in contact with the light source 320. The protection layer also provides a surface that may be readily cleaned and/or sanitized. The protection layer is preferably optically transparent so as to allow the light emitted by the light source 320 to pass through the protection layer and avoid any heating during operation of the lamp 300. In some instances, the protection layer may be made of one or more thermoplastic polymers such as polycarbonates. The protection layer may cover the entirety of the light source and may be affixed on the light-emitting side 302 of the lamp 300 through any suitable securing means (e.g., screws).

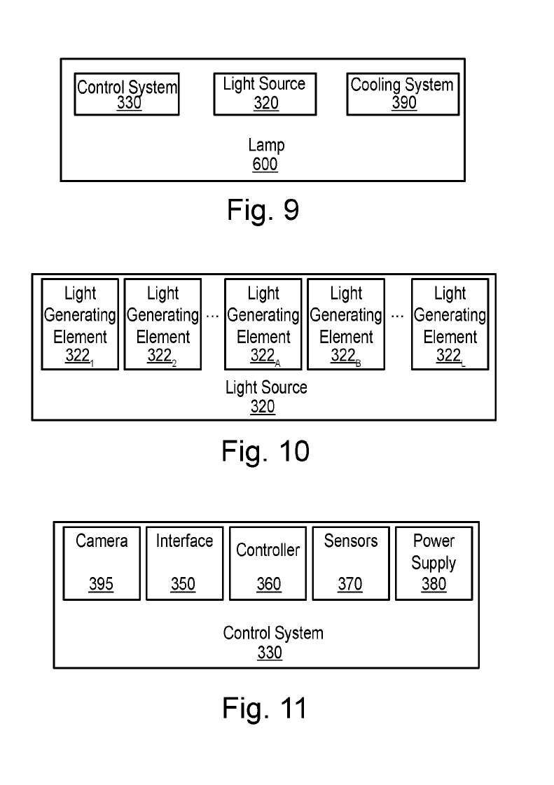

The light source 320 may be implemented by any source of light that can emit light having characteristics desirable for the phototherapy treatment on the subject 200. In the present embodiment of the configurable phototherapeutic device 100, each light source 320 is made up of many light-emitting diodes (LEDs) 322₁-322_{L} (see FIG. 9). The light source 320 may be implemented in any other suitable way in other embodiments. For example, in other embodiments, the light source 320 may include, but is not limited to, a single light-generating element and/or may be implemented using any other type of light-generating element, such as, for instance, an incandescent light bulb, an electron-stimulated luminescence element such as an electroluminescent material (e.g., an electroluminescent wire or sheet), a field-induced polymer electroluminescent material, a gas-discharge bulb such as a fluorescent lamp, a cathode lamp, a neon or argon lamp, a plasma lamp, a xenon flash lamp, a high-intensity discharge lamp such as a metal-halide lamp, an active-matrix organic light-emitting diode (AMOLED), an organic light-emitting diode (OLED), a semi-conductor (diode) laser, a fiber laser, a solid-state laser, diode-pumped solid-state laser, a photonic crystal laser, an arc discharge, or the like. One skilled in the art would understand that any other light source could be used within the scope of the present technology and would not be restricted to the examples listed above, so long as it achieves the desired effect of the phototherapy treatment. In various examples of implementation, the light emitted by the light source 320 may be pulsed or continuous, and may be spectrally concentrated (i.e., narrowband) or spectrally diffuse (i.e., broadband). The light emitted by the light source 320 may have any other suitable characteristics for the phototherapy treatment on the subject 200 in other embodiments.

With additional reference to FIG. 10, in this embodiment, the light source 320 comprises a plurality of light-generating elements 322₁-322_{L} that collectively emit the light for the phototherapy treatment on the subject 200. As mentioned above, in this embodiment, the light-generating elements 322₁-322_{L} are light-emitting diodes (LEDs) 322₁-322_{L}.

The light emitted by the light source 320 may have any suitable characteristics for the phototherapy treatment on the subject 200. In this embodiment, the characteristics of the light emitted by the light source 320 may be selected based on the photoactivatable composition 600 with which the phototherapeutic device 100 may be used.

In some embodiments, the light-generating elements 322₁-322_{L} may be configured to each emit the same type of light. In other embodiments, the light-generating elements 322₁-322_{L} may be split into two or more groups, with each group emitting the same type of light. In such embodiments, a therapeutic effect of the light emitted by a first group of the light-generating elements 322₁-322_{L} may be augmented and/or complemented by a therapeutic effect of another group of the light-generating elements 322₁-322_{L}. Here, the “types” of light may be differentiated based on a number of factors, including peak wavelength, average wavelength, bandwidth, intensity, or the like.

Similarly, the light sources 320 of the different lamps 300 of the phototherapeutic device 100 may be configured to each emit the same type of light. In other embodiments, the light sources 320 may be split into two or more groups, with each light source 320 within one group emitting the same type of light, and the groups of light sources 320 emitting different types of light from one another, such as the phototherapeutic patterns above. As described above, the central controller 175 selectively causes the light sources 320 of the lamps 300 to emit the required type of light for each group or phototherapeutic pattern.

For instance, in some examples, the peak wavelength of either group or for some or all of the lamps 300 may be about 430 nm to about 500 nm, about 440 nm to about 500 nm, about 450 nm to about 500 nm, about 430 nm to about 475 nm, about 435 nm to about 470 nm, about 440 nm to about 460 nm, about 440 nm, about 450 nm, about 460 nm, or about 470 nm. In some alternative embodiments, the peak wavelength may be about 480 nm to about 760 nm, about 480 nm to about 700 nm, about 480 nm to about 650 nm, about 480 nm to about 620 nm, about 500 nm to about 700 nm, about 520 nm to about 700 nm, about 500 nm to about 660 nm, about 540 nm to about 640 nm, about 540 nm to about 580, about 500 nm to about 570 nm, about 570 nm to about 590 nm, about 590 nm to about 610 nm, about 610 nm to about 760 nm, or may be within the infrared range of the electromagnetic spectrum.

In certain embodiments, an average power density of the light emitted by one or more of the light sources 320 and/or received by the treatment sites 210_{X}, measured at a treatment distance, is less than about 200 mW/cm², about 4 mW/cm² to about 75 mW/cm², about 15 mW/cm² to about 75 mW/cm², about 10 mW/cm² to about 200 mW/cm², about 10 mW/cm² to about 150 mW/cm², 20 mW/cm² to about 130 mW/cm², about 55 mW/cm² to about 130 mW/cm², about 90 mW/cm² to about 140 mW/cm², about 100 mW/cm² to about 140 mW/cm², or about 110 mW/cm² to about 135 mW/cm². In some embodiments, an average power density of the light emitted by the light source 320 is about 4 mW/cm² to about 75 mW/cm², about 10 mW/cm² to about 75 mW/cm², about 10 mW/cm² to about 85 mW/cm², about 15 mW/cm² to about 75 mW/cm², about 30 mW/cm² to about 70 mW/cm², about 40 mW/cm² to about 70 mW/cm², about 55 mW/cm² to about 85 mW/cm2², or about 55 mW/cm² to about 65 mW/cm².

In certain embodiments, an average fluence emitted by the light source 320 and/or received by the treatment site 210, during a single phototherapy treatment procedure, is at least about 0.01 J/cm², or is more than about 4 J/cm², more than about 10 J/cm², more than about 15 J/cm², more than about 30 J/cm², more than about 50 J/cm², up to about 60 J/cm². In certain embodiments, the light sources 320 could be configured to emit light having a fluence, during a single treatment, of about 4 J/cm² to about 60 J/cm², about 10 J/cm² to about 60 J/cm², about 10 J/cm² to about 50 J/cm², about 10 J/cm² to about 40 J/cm², about 10 J/cm² to about 30 J/cm², about 20 J/cm² to about 40 J/cm², about 15 J/cm² to about 25 J/cm², or about 10 J/cm² to about 20 J/cm².

The control system 330 is configured to control operation of each lamp 300, including the light source 320, and to provide information relating to operational parameters of the lamp 300 to the central controller 175 or the user of the configurable phototherapeutic device 100. For example, the control system 330 may turn on and off the light source 320, control (e.g., start, stop, modulate) emission of light (activate/deactivate certain LEDs, or the like) by the light source 320, control the cooling system 390 (e.g., turn on or off a fan, or the like), monitor parameters while the phototherapy treatment is being performed on the subject 200, cause information to be output to the user, and/or perform any other action. The control system 330 comprises suitable hardware and/or software (e.g., firmware) configured to implement its functionality. In some embodiments, one or more of the lamps 300 could be provided without some or all of the control system 330 or its components, and their functions, in whole or in part, could be replaced or managed by the central controller 175.

With additional reference to FIG. 11, in this embodiment, the control system 330 comprises an interface 350, a controller 360, a camera 395, and one or more sensors 370. The control system 330 also comprises a power supply 380, which provides power to the lamp 300, including, in this case, to the light source 320 and the cooling system 390.

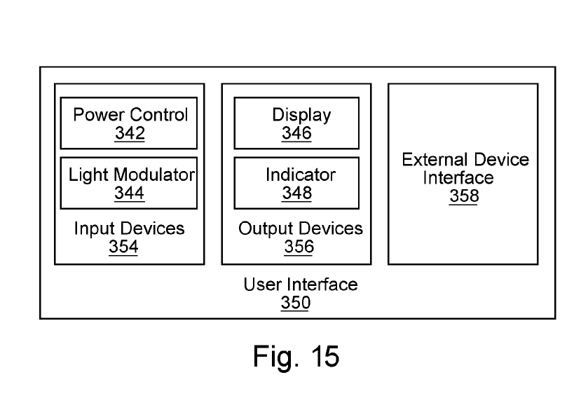

The interface 350 is configured to receive inputs, including commands, signals, and/or other information, and issue outputs, including commands, signals and/or other information. With additional reference to FIG. 15, in this embodiment, the interface 350 comprises a user interface 351 and an external device interface 358.

The user interface 351 is configured for receiving inputs from and outputting information to the user of the configurable phototherapeutic device 100. To this end, in this example, the user interface 351 may include one or more input devices 354 and one or more output devices 356. In some embodiments, the output devices 356 may be communicatively connected to the display 190 to allow the user of the phototherapeutic device 100 to monitor one or more operational parameters of one or more of the lamps 300. The user interface 351 may include any number of additional and/or different input devices 354 and/or output devices 356. In some embodiments, one or more of the lamps 300 could be provided without some or all of the interface 350 or its components, and their functions, in whole or in part, could be replaced by the central controller 175.

In this embodiment, the external device interface 358 allows an external device (e.g., a computer such as a server, a laptop computer, a desktop computer, a tablet, or another computing device; a smartphone or another mobile communication device; a portable memory stick, or the like) to connect to the lamp 300. As above, in some embodiments individual lamps 300 may not include the external device interface 358. Additionally or alternatively, the central controller 175 may include the external device interface 358 in order to provide communication and connection with an external device (not shown) with the configurable phototherapeutic device 100.

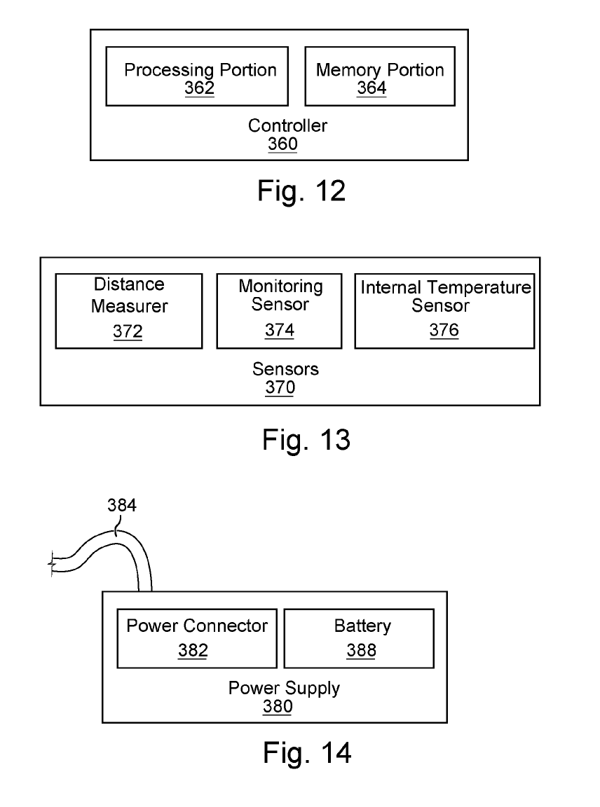

With additional reference to FIGS. 11 and 12, the controller 360 is communicatively coupled to the interface 350 and comprises a processing portion 362 and a memory portion 364.

The processing portion 362 comprises one or more processors to perform processing operations that implement functionality of the controller 360. A processor of the processing portion 362 may be a general-purpose processor executing program code stored in the memory portion 364. Alternatively, a processor of the processing portion 362 may be a specific-purpose processor comprising one or more preprogrammed hardware or firmware elements (e.g., application-specific integrated circuits (ASICs), electrically erasable programmable read-only memories (EEPROMs) or other related elements).

The memory portion 364 comprises one or more memories for storing program code executed by the processing portion 362 and/or data used during operation of the processing portion 362. A memory of the memory portion 364 may be implemented as a semiconductor medium (including, e.g., a solid state memory), a magnetic storage medium, an optical storage medium, and/or any other suitable type of memory. A memory of the memory portion 364 may be read/writeable, read-only (ROM), and/or random-access (RAM). Of course, other implementations of the memory portion 364 are possible.

In this embodiment, the controller 360 comprises one or more printed circuit boards (PCBs) 355 implementing at least part of the processing portion 362 and the memory portion 364 and exchanging control signals with other components of the lamp 300, including with the light source 320 to control emission of light by the light source 320.

As before, in some embodiments, the lamps 300 could be provided without the processing portion 362 and the memory portion 364 of the controller 360 and their functions, in whole or in part, could be replaced by the central controller 175.

With additional reference to FIG. 13, in this embodiment, the control system 330 of each lamp 300 also comprises the one or more sensors 370 that are communicatively coupled to the interface 350 to provide to the controller 360 and the central controller 175 various information regarding a state of the lamp 300 and/or, more generally, conditions and progress of the phototherapy treatment. It is again contemplated that the sensors 370 may be excluded from some or all of the lamps 300, depending on the embodiment.

In this embodiment, the sensors 370 of each lamp 300 include a distance measurer 372 to measure a distance Cₛ between the light-emitting side 302 of the lamp 300 and the subject 200 (as illustrated in FIG. 5). This may help to have a proper or optimal spacing between each lamp 300 and the subject 200 for treatment purposes. The sensors 370 also include a monitoring sensor 374 for monitoring the progress of a phototherapy treatment procedure.

Some embodiments of the control system 330 may comprise additional and/or different sensors. These may include infrared sensors, laser-based sensors, high-frequency radio-wave sensors, radar or other sound-based sensors, or any other suitable type of sensor, with further details being provided in the following paragraphs.

The distance measurer 372 is configured for sensing the distance Cₛ between the light-emitting side 302 of the lamp 300 and the subject 200. The distance measurer 372 may implement one or more known range-finding techniques, including laser-based distance measurement, infrared-based distance measurement, visible-light-based distance measurement, or any other suitable range-finding technique. Other implementations of the distance measurer 372 may also be possible. The distance measurer 372 may be used to provide an indication of the distance Cₛ between the light-emitting side 302 of the lamp 300 and the subject 200 to the user or the central controller 175. In some embodiments, an indicator, such as a visual indicator such as an LED or a sound-emitting device, could be used to convey information from the distance measurer 372.

Alternatively or additionally, an indication may be sent to the central controller 175 that the distance Cₛ is too large or too small relative to a predetermined reference distance between one of the lamps 300 and the subject 200 (which may depend on the light emitted, type of treatment, or the like). For example, in some embodiments, a notification, such as a textual message (e.g., “lamp too close”, “lamp too far”, “lamp properly positioned”, or the like), a graphical icon (e.g., an arrow pointing towards or away from a human representation), and/or another message, may be conveyed on the display 190 connected to the central controller 175.

In some embodiments, the control system 330 of each lamp 300 may be configured to control the emission of light by the lamp 300 based on the distance Cₛ between the light-emitting side 302 of the lamp 300 and the subject 200. Additionally or alternatively, the central controller 175 may be configured to control the emission of light by one or more of the lamps 300 based on one or more distances Cₛ between the light-emitting side 302 of one or more of the lamps 300 and the subject 200. In the event that one or more of the distances Cₛ is unsuitable for the treatment, relative to a predetermined reference distance, the control system 330 or the central controller 175 may take an action accordingly. For example, the control system 330 or the central controller 175 may be configured to convey an indication of the unsuitability of the distance Cₛ to the subject 200 to be treated, for example by way of the display 190 of the central controller 175. As another non-limiting example, the central controller 175 may be configured to decrease the intensity of the light emitted by one or more of the lamps 300 or to substantively turn off one or more of the lamps 300 in response to one or more of the distances Cₛ being unsuitable.

In this embodiment, the monitoring sensor 374 is configured for monitoring the irradiated area 220 which is irradiated by the light emitted by the lamp 300 of the phototherapeutic device 100 to provide an indication of a level of progress of the phototherapy treatment that may allow the controller 360, or the central controller 175, to control operation of the lamp 300 based on the indication of the level of progress provided by the monitoring sensor 374. In this example, the irradiated area 220 may intersect the photoactivatable compositions 600x and/or the treatment sites 210_{X} of the subject 200, as discussed above. The monitoring sensor 374 may be an optical sensor, a thermal sensor, a camera, or any other suitable sensing mechanism.

In some embodiments, two or more sensors 370 of the control system 330, such as the distance measurer 372 and the monitoring sensor 374, may be implemented as the same sensor. In other embodiments, two or more sensors 370 of the control system 330, such as the distance measurer 372 and the monitoring sensor 374, may share at least some components.

As there are more than one or many lamps 300 in the configurable phototherapeutic device 100, each lamp 300 thereof needs not contain all pertinent sensors, etc. In some embodiments, for example, the monitoring sensor 374 could be implemented in one lamp 300, while the distance measurer 372 could be implemented in a neighboring lamp 300. As before, in some embodiments, the lamps 300 could be provided without the any of the sensors, and their functions, in whole or in part, could be replaced by the central controller 175. It is also contemplated that the central controller 175 could be operatively connected to external sensors (not shown).

With additional reference to FIG. 14, the power supply 380 supplies power to operate the lamp 300. In this embodiment, the power supply 380 comprises a power connector 382 for connecting to an external power source (not pictured). In this example, the power connector 382 is connectable to the external power source via a power cable 384 which may route or otherwise provide power from the external power source to the power supply 380. The power cable 384 may be any suitable cable, including, for example, a cable satisfying one or more of the IEC 60320 standards, such as the so-called IEC cord. In some embodiments the power cable 384 may connect to an electrical system internal to the configurable phototherapeutic device 100 (not shown). In other examples, the power connector 382 may be an inductive charging interface which may be inductively coupled to a charging station (not pictured) and may be configured to convert an electromagnetic field generated by the charging station into electrical power supplied to the lamp 300.

In this embodiment, the power supply 380 includes a battery 388. The battery 388 may be implemented using any suitable battery technology, including lead-acid, nickel-cadmium, nickel-metal-hydride, lithium-ion (including lithium-polymer, lithium-iron-phosphate, lithium-titanate) and the like. The battery 388 may act as a primary power source for the lamp 300, such that the lamp 300 may be capable of operating for extended periods of time relying solely on the power provided by the battery 388, and may only need to be recharged by the external power source periodically. In other embodiments, the battery 388 may act as a backup power source, preventing the lamp 300 from turning off in the event of a failure of the external power source. In still further embodiments, the battery 388 may at times act as the primary power source and at other times act as the secondary power source.

It is contemplated that in some embodiments, the lamps 300 could be provided without the power supply 380 or parts thereof, and power could be supplied by an electrical power source within the configurable phototherapeutic device 100 (not shown) or via the electrical connection between the lamps 300 and the central controller 175.

In some embodiments, with additional reference to FIG. 11, the control system 330 of each lamp 300 may comprise a camera 395 to capture images of the irradiated area 220 in order to acquire visual information about the irradiated area 220, which may include visual information about the treatment site 210 of the subject 200 and/or visual information about the photoactivatable composition 600. These images may be viewed and/or processed during the phototherapy treatment and/or subsequently.

In some examples of implementation, the camera 395 may be one of the sensors 370 of the control system 330, or may activate one or more of the sensors 370, such as the distance measurer 372 and/or the monitoring sensor 374. In other examples of implementation, the camera 395 may be separate from any other sensor that one or more of the lamps 300 may include.

In some embodiments, the images captured by the camera 395 may be presented to the user, for example, on the display 190 of the central controller 175. As before, in some embodiments, all or some of the lamps 300 could be provided without the camera 395 and their functions, in whole or in part, could be replaced by the central controller 175. It is also contemplated that the central controller 175 could communicate with and control one or more of the cameras 395 of the lamps 300.

Additionally or alternatively, in some embodiments, the images captured by the camera 395 may be stored in a storage device, such as, for example, in the memory portion 364 of the controller 360, a memory portion in the central controller 175 (not shown) or in an external storage device (not shown) that is external to the configurable phototherapeutic device 100. The stored images may then be retrieved at a later time for viewing or processing via the phototherapeutic device 100 or via an external computing device. For instance, in some cases, the images may be retrieved during or soon after completion of the phototherapy treatment on the subject 200. In other cases, the images may be retrieved a significant period of time (e.g., one or more days, weeks or months) after completion of the phototherapy treatment on the subject 200.

In some embodiments, the one or more cameras 395 of the lamps 300 may capture images continuously over a certain period, as video. In other embodiments, the cameras 395 may capture images at specified moments in time, such as periodically or when a certain condition is fulfilled (e.g., at a beginning, midpoint, and end of the phototherapy treatment procedure on the subject 200).

In addition to and/or instead of being presented for viewing, in some embodiments, images captured by one or more of the cameras 395 may be processed by the central controller 175 to control operation of one or more of the lamps 300. For example, in some embodiments, images captured by one of the cameras 395 could be processed to detect changes in the light emitted by one or more of the lamps 300, such as changes in intensity or peak or average wavelength, in the photoactivatable composition 600, such as changes in color, position, shape, reflectivity, transmissivity, and the like, or in one of the treatment sites 210 of the subject 200, such as reddening. In response to detecting such changes, the central controller 175 may perform an action relating to controlling one or more of the lamps 300, such as change (e.g., decrease or increase) the intensity of light emitted by the lamp 300 (e.g., turn off the lamp 300), or the like. In some examples, upon detecting such changes, or any other suitable changes, one or more of the cameras 395 may be operative to acquire one or more images.

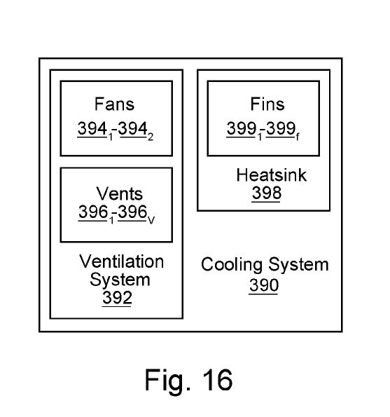

With reference to FIGS. 7 and 16, the cooling system 390 helps dissipate heat generated by the light source 320 and the control system 300. In this embodiment, the cooling system 390 comprises a ventilation system 392 and a heatsink 398 which may be exposed to air circulated by the ventilation system 392.

More particularly, in this embodiment, the heatsink 398 is adjacent to the light source 320 to conduct heat from the light source 320 and dissipate that heat by convection. In this example, the heatsink 398 comprises a plurality of fins 399₁-399_{f}, which are elongated in the longitudinal direction of the lamp 300 and reach toward a longitudinal end 304 of the lamp 300. Also, in this embodiment, the ventilation system 392 comprises a plurality of fans 394₁-394₂ and a plurality of vents (or ventilation openings) 396₁-396ᵥ.

In this example, the fan 394₁ is configured to direct airflow toward at least part of the control system 330, which is implemented on the PCB 355. More specifically, by directing this airflow, the fan 394₁ may effect a dissipation of heat from the PCB 355 via convection. In this case, the fan 394₁ is located and exposed at the periphery 314 of the housing 312 adjacent to the PCB 355. Additionally, the fan 394₁ is positioned such that a projection of the fan 394₁ parallel to an axis of rotation of the fan 394₁ intersects the PCB 355. Also, in this example, the fan 394₂ is configured to direct airflow toward the heatsink 398 thereby cooling at least part of the heatsink 398. More specifically, by directing this airflow, the fan 394₂ may effect a dissipation of heat from the heatsink 398 via convection. In this case, the fan 394₂ is configured to direct the airflow along and/or between the fins 399₁-399_{f}.

In this embodiment, the fans 394₁-394₂ are oriented differently from one another. Put differently, their respective axis of rotation are transversal (i.e., nonparallel) to one another. In some such embodiments, the respective axis of rotation of each of fans 394₁-394₂ may be substantively perpendicular to one another. In some cases, the particular orientation of the fans 394₁-394₂ may induce a certain amount of turbulence, which may help to more efficiently dissipate heat by convection.

The vents 396₁-396ᵥ are configured to allow air to flow in and out of the housing 312 of the lamp 300. The vents 396₁-396ᵥ are located on the periphery 314 of the frame 310, and may be configured so as to permit airflow between the cavity 319 and an exterior environment in which the phototherapeutic device 100 operates. For example, in this embodiment, at least one of the vents 396₁-396ᵥ is located adjacent to the fan 394₁. Also, in this embodiment, at least one of the vents 396₁-396ᵥ is located at the longitudinal end 304 of the housing 312 where the fins 399₁-399_{f} terminate. In some embodiments, each of the lateral surfaces 317₁-317₄ may comprise at least one vent. In other embodiments, only some of the lateral surfaces 317₁-317₄ may comprise at least one vent. In some embodiments, the vents 396₁-396ᵥ may only be located on those lateral surfaces which do not have any one of the handles 318₁-318₃. The vents 396₁-396ᵥ may be arranged in any other suitable manner in other embodiments.

In certain implementations of this embodiment, the housing 312 comprises means (not shown) for directing the flow of air exiting fan 394₁, such as by, but not limited to, air ventilation flaps and/or adjustable rings comprising one or more portals for directing the flow of the air exiting fan 394₁. The direction of the air ventilation flaps and/or portal-bearing rings may be adjustable (using, for example, a knob) so as to deflect the flow of air exiting fan 394₁ away from the subject (e.g., patient). The means for directing the flow of air exiting fan 394₁ may be mounted on top of fan 394₁ and may be affixed onto surface 317₄ of the housing 312.

The control system 330, and more specifically, the controller 360, is configured to control the fans 394₁-394₂. In some embodiments, the controller 360 may control the fans 394₁-394₂ independently from one another. For example, in some embodiments, the fan 394₂ may be more active, or may be active for longer periods of time, than the fan 394₁, since the light source 320 may generate more heat than the PCB 355. In some embodiments, the controller 360 may be operative to activate, deactivate, or otherwise control the action of the fans 394₁-394₂ based on one or more conditions, including for example a temperature sensed by an internal temperature sensor 376. The controller 360 may be operative to control the action of the fans 394₁-394₂ based on whether or not one or more temperatures in the cavity 319 exceed their respective allowed temperature thresholds.

In some embodiments, a speed of rotation of each of the fans 394₁-394₂ may be controlled by the controller 360 based on one or more of the temperatures and/or based on a difference between one or more of the temperatures and their respective allowed temperature threshold. In embodiments of the lamps 300 lacking an internal temperature sensor 376, the controller 360 may be configured to monitor a time of operation of the lamp 300 and may control the action of the fans 394₁-394₂ (such as, e.g., increasing their respective speed of rotation) based at least in part on the time of operation. Other factors may be considered by the controller 360 when controlling the action of the fans 394₁-394₂ including, but not limited to, an intensity of the light source 320, an indication of the number of sensors 370 in use, and an indication of whether the lamp 300 is receiving power from an external power source via the power connector 382, from the battery 388, or from both.

In some embodiments, the central controller 175 could include similar cooling systems as those described above. Additionally or alternatively, the central controller 175 could control the fans 394₁-394₂ or generally replace the controller 360 of some or all of the lamps 300.

With reference to FIG. 17, a phototherapeutic device 102 according to another embodiment of the present technology is illustrated. The phototherapeutic device 102 includes a support structure 150 for supporting the lamps 300. The lamps 300 are movably connected to the support structure 150 such that they may be angled to appropriately direct light emitted by the lamps 300 toward one or more anatomical features of the subject 200. Unlike the phototherapeutic device 100, described above, the lamps 300 cannot be shifted or displaced with respect to one another.

In some embodiments of the phototherapeutic device 102, one or more of the lamps 300 could be selectively detachably connected to the support structure 150, such that the lamps 300 are selectively removable from and reconnectable to the support structure 150. In some embodiments, one or more lamps 300 from the phototherapeutic device 100 may be removable from the support structure 150 and usable as a stand-alone device by the user. Remaining portions of the phototherapeutic device 102 are identical to the phototherapeutic device 100 described above and as such need not be repeated here.

With reference to FIG. 18, a configurable phototherapeutic device 104 according to another embodiment of the present technology is illustrated. The phototherapeutic device 104 includes a support structure 150 for supporting the lamps 300. Unlike the phototherapeutic devices 100 and 102, the lamps 300 of the phototherapeutic device 104 cannot spatially adjusted. The central controller 175, however, can still selectively activate different groups of lamps 300 in order to produce patterns to provide concurrent, different phototherapy treatments.

Further, the support structure 150 includes a top portion 198 and a bottom portion 199 which are generally shaped to direct light from the lamps 300 toward one or more anatomical features of the subject 200. The top portion 198, as shown in an open position, allows the subject 200 to enter into the phototherapeutic device 104 and lie down on the bottom portion 199. In a closed position (not shown), the subject 200 is surrounded by the lamps 300, which are then controlled by the central controller 175 to provide one or more concurrent phototherapy treatments. Remaining portions of the phototherapeutic device 104 are identical to the phototherapeutic device 102 described above and as such need not be repeated here.

While the support structure 150 is shown with the top portion 198 disposed above the bottom portion 199, it is contemplated that the support structure 150 could be differently oriented, such that the subject 200 to be treated could enter vertically, with the top and bottom portions 198, 199 disposed on the right and left sides of the subject 200 respectively.

With reference to FIG. 19, a configurable phototherapeutic device 106 according to yet another embodiment of the present technology is illustrated. The configurable phototherapeutic device 106 is a portable embodiment of the present technology, designed for over-the-counter use by the subject 200 in the comfort of their own home. In some implementations of the portable embodiment, the configurable phototherapeutic device 106 could also be transported to the home of the subject 200 to be used by a health care provider. As is illustrated in FIG. 19, the configurable phototherapeutic device 106 is relatively small and can be used for concurrent phototherapy treatment of a small area of anatomical features, in this example: the fingers, the palm, and the back of the hand of the subject 200, although the device 106 is suited for treatment of many other anatomical features.

The configurable phototherapeutic device 106 includes three small lamps 300 adjustably connected together via the connecting members 157 (shown schematically). The connecting members 157 are hinged, such that the three lamps 300 can be angled to create different treatment configurations. For example, the three lamps 300 could be arranged parallel to one another, creating one larger, flat panel. In another non-limiting example, the user could close the lamps 300 around an anatomical feature, similar to the example illustrated in FIG. 19, such that all sides of the anatomical feature to be treated (such as the hand) are aligned with at least a portion of one of the lamps 300. It is contemplated that the configurable phototherapeutic device 106 could include more or less lamps 300 and/or connecting members 157, depending on the desired size, shape and effect of the device 106. In the embodiment illustrated, there is no support or base structure to hold the lamps 300, and the user could hold the device 106 with one hand, or prop the device up on a solid surface, for example a table. In some non-limiting embodiments, the configurable phototherapeutic device 106 could include a base structure to hold the lamps 300 in the desired configuration and orientation. In some non-limiting embodiments, the configurable phototherapeutic device 106 could be provided with a stand for receiving and supporting the device 106.

The central controller 175 is a module with wireless connectivity integrated into one of the lamps 300 (shown schematically). The central controller 175 receives instructions to operate the device 106 wirelessly from an electronic device 299 of the subject 200. The electronic device 299 of the subject 200 could include, but is not limited to, a personal computer, a smart phone, a tablet, a server, and any other suitable device. Use of the electronic device 299 to aid in operation of the device 106 contributes in part to the mobility and transportability of the configurable phototherapeutic device 106. The configurable phototherapeutic device 106 could include an application 298 to be installed on the electronic device 299 of the subject 200, as is shown schematically in FIG. 19. It is contemplated that the application 298 for operating the configurable phototherapeutic device 106 via the central controller 175 could also be available for download via a third-party supplier. It is contemplated that the central controller 175 could be differently implemented, for instance by including a touch-screen interface on a rear side of one of the lamps 300 (not shown). In some embodiments where the device 106 is designed for over-the-counter use, controls provided by the application 298 may be highly simplified and designed such that any subject 200, even a child, could operate the configurable phototherapeutic device 106.

The three lamps 300 of the configurable phototherapeutic device 106 can be folded onto one another in an overlapping fashion to create a generally rectangular, flat shape. This allows the device 106 to be packaged in, for example, a packaging unit such as a box, convenient for sale to users, such as the subject 200 himself/herself. It is contemplated that the configurable phototherapeutic device 106 could be sold as a kit, the kit further including instructions for use and/or setup and/or installation of the device 106. In some embodiments, the kit including the configurable phototherapeutic device 106 could further include various related elements, including, but not limited to: a delivery system for one or more photoactivatable compositions 600, a stand or support structure for receiving and holding the device 106, a power supply, and cables for connecting to a power supply or power source.

Remaining portions of the phototherapeutic device 106 are identical to the phototherapeutic device 100 described above and as such need not be repeated here.

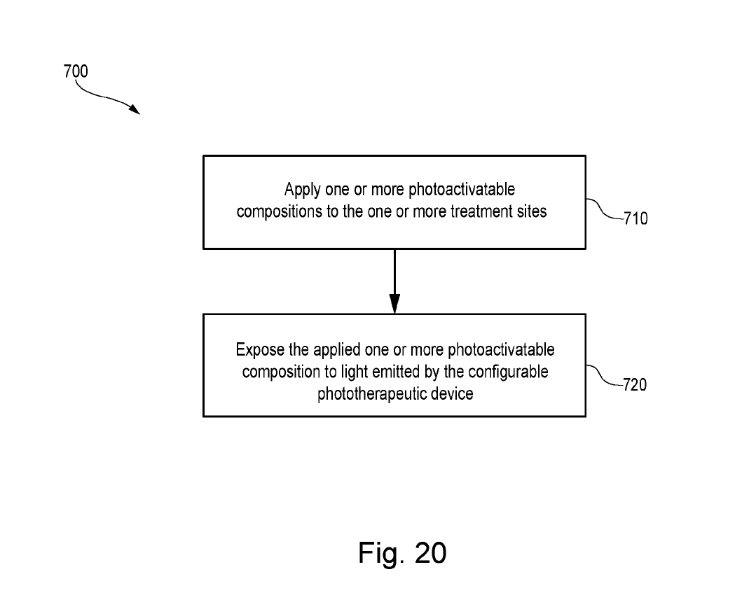

With reference to FIG. 20, a method 700 of using the configurable phototherapeutic device 100 will be described. The method 700 comprises, at step 710, first applying the one or more photoactivatable compositions 300_{X} to the one or more treatment sites 210_{X}. The method 700 then continues at step 720 with exposing the applied photoactivatable compositions 300_{X} to light emitted by the lamps 300 of the configurable phototherapeutic device 100 to activate the one or more photoactivatable compositions 300_{X}, where activation of the one or more photoactivatable compositions 300_{X} allows treatment of the one or more treatment sites 210_{X}. In some implementations, a first portion of the one or more photoactivatable compositions 300_{X} is exposed to a first phototherapeutic pattern and at least a second portion of the photoactivatable compositions 300_{X} is concurrently exposed to a second phototherapeutic pattern.

While in embodiments considered above the subject 200 is a human, the subject 200 treated by the configurable phototherapeutic devices 100, 102, 104, and/or 106 may be another animal or any other organism. For example, it is contemplated that the configurable phototherapeutic systems or devices 100, 102, 104, 106 could be implemented in an animal setting, where the subject 200 is an animal such as, but not limited to: a cat, dog, horse, cow, sheep, and the like. In such a case, the animal subject to be treated may be anesthetized in order to temporarily restrict the movement of the animal subject. It is also contemplated that the animal subject could be, additionally or alternatively, situated in an immobilizing unit such as a gated chute or other suitable type of immobilizing apparatus in order to temporarily restrict the movement of the animal subject. It is also contemplated that the configurable phototherapeutic systems or devices 100, 102, 104, 106 could include any suitable type of immobilizing apparatus for use in an animal setting.

As one non-limiting example, the subject 200 could be a horse being treated using the configurable phototherapeutic device 100. In one example implementation, the configurable phototherapeutic device 100 could be used to provide concurrent phototherapy treatment to multiple treatment sites along the leg of the horse. By adjusting and orienting the lamps 300 around the leg of the horse using the adjustable connecting members 157, the multiple treatment sites along the leg of the horse could receive different and concurrent phototherapy treatment. This is one non-limiting example of an implementation of the configurable phototherapeutic device 100. It is contemplated that the configurable phototherapeutic device 100, or other embodiments thereof, could be used to treat different anatomical features of a horse, such as the head or back. It is also contemplated that the configurable phototherapeutic device 100, or other embodiments thereof, could be configured to provide different concurrent phototherapy treatments to other animals as well, including but not limited to: cats, dogs, other household pets, apes, rodents, and livestock animals.

Any feature of any embodiment discussed herein may be combined with any feature of any other embodiment discussed herein in some examples of implementation. Certain additional elements that may be needed for operation of certain embodiments have not been described or illustrated as they are assumed to be within the purview of those skilled in the art. Moreover, certain embodiments may be free of, may lack and/or may function without any element that is not specifically disclosed herein.

Modifications and improvements to the above-described implementations and embodiments of the present technology may become apparent to those skilled in the art. The foregoing description is intended to be exemplary rather than limiting. The scope of the present technology is therefore intended to be limited solely by the scope of the appended claims.

1. A configurable photo therapeutic device comprising:
(a) a support structure;
(b) a plurality of light emitting devices adaptably connected to the support structure, the plurality of light emitting devices being configured for providing concurrent phototherapy to a plurality of anatomical features of a subject, each light emitting device of the plurality of light emitting devices comprising at least one light source configured to emit light adapted for phototherapy; and
(c) a central controller operatively connected to the plurality of light emitting devices, each one of the plurality of light emitting devices being communicatively connected to the central controller, the central controller being configured for selectively causing at least one of the plurality of light emitting devices to emit light for phototherapy of at least one anatomical feature of the plurality of anatomical features of the subject, and the support structure being adapted for directing the light emitted by the at least one of the plurality of light emitting devices toward the at least one anatomical feature of the subject.

2.-3. (canceled)

4. The configurable phototherapeutic device as defined in claim 1, wherein the central controller is configured to provide one or more phototherapeutic patterns of emitted light by selectively activating at least one subset of the plurality of light emitting devices.

5. The configurable phototherapeutic device as defined in claim 4, wherein the one or more phototherapeutic patterns provide concurrent phototherapy for one or more conditions of the subject.

6. The configurable phototherapeutic device as defined in claim 4, wherein the central controller is configured to: activate a first subset of the plurality of light emitting devices to provide a first phototherapeutic pattern; and activate a second subset of the plurality of light emitting devices to provide a second phototherapeutic pattern.

7. The configurable phototherapeutic device as defined in claim 6, wherein the central controller is further configured to activate a third subset of the plurality of light emitting devices to provide a third phototherapeutic pattern.

8. The configurable phototherapeutic device as defined in claim 7, wherein the central controller is further configured to activate a fourth subset of light emitting devices to provide a fourth phototherapeutic pattern.

9. The configurable phototherapeutic device as defined in claim 8, wherein the central controller is further configured activate a fifth subset of the plurality of light emitting devices to provide a fifth phototherapeutic pattern.

10. The configurable phototherapeutic device as defined in claim 4, wherein light emitted in the one or more phototherapeutic patterns differ in wavelength of light emitted.

11. The configurable phototherapeutic device as defined in claim 1, wherein light emitted in the one or more phototherapeutic patterns differ in power density of light emitted.

12. The configurable phototherapeutic device as defined in claim 4, wherein a first subset of the light emitting device emits light with a first peak wavelength, a second subset of the light emitting devices emits light with a second peak wavelength, and a third subset of the light emitting devices emits light with a third peak wavelength.

13.-14. (canceled)

15. The configurable phototherapeutic device as defined in claim 1, wherein the configurable phototherapeutic device is adaptable to the contour of the one or more anatomical features of the subject.

16. The configurable phototherapeutic device as defined in claim 1, wherein the first phototherapeutic pattern is in therapeutical alignment with a first anatomical feature of the subject.

17. The configurable phototherapeutic device as defined in claim 6, wherein the second phototherapeutic pattern is in therapeutical alignment with a second anatomical feature of the subject.

18. The configurable photo therapeutic device as defined in claim 1, each one of the plurality of light emitting devices comprising:
a light source for emitting light adapted for phototherapy;
a light emitting device control system for controlling operation of the light emitting device; and
a cooling system for cooling the light emitting device.

19. The configurable photo therapeutic device as defined in claim 17, wherein the light emitting device control system is operative to acquire information relating a position of the light emitting device relative to the at least one anatomical feature of the subject.

20. The configurable phototherapeutic device as defined in claim 1, wherein each one of the plurality of light emitting devices further comprise a sensor operatively connected to the light emitting device controller.

21. The configurable phototherapeutic device as defined in claim 20, wherein the sensor is configured to provide information relating to at least one of: an operational state of the light source; one or more conditions of an environment surrounding the light emitting device; and progression of the phototherapy.

22. The configurable phototherapeutic device as defined in claim 20, wherein each sensor of the plurality of light emitting devices is operatively connected to the central controller.

23. The configurable phototherapeutic device as defined in claim 17, wherein the light emitting device control system of each of the plurality of light emitting devices controls emission of light by the light source based on a distance between the light emitting device and the at least one anatomical feature of the subject.

24. The configurable phototherapeutic device as defined in claim 23, wherein the sensor is configured to determine the distance between the light emitting device and the at least one anatomical feature of the subject.

25.-87. (canceled)
